(19) United States
(12) Patent Application Publication
(10) Pub. No.: US 2017/0103636 A1
(43) Pub. Date: Apr. 13, 2017

(54) IMPROPER SPINAL CURVATURE WARNING APPARATUS

(71) Applicant: FENG CHING TU, TAIPEI CITY (TW)

(72) Inventor: FENG CHING TU, TAIPEI CITY (TW)

(21) Appl. No.: 14/877,174

(22) Filed: Oct. 7, 2015

(51) Int. Cl. G08B 21/04; A61B 5/107; A61B 5/00

(52) U.S. Cl. CPC G08B 21/0446; A61B 5/407; A61B 5/7455; A61B 5/7405; A61B 5/1077

(57) ABSTRACT

An improper spinal curvature warning apparatus includes: a main body having a casing and a patch installed under the casing, a casing port formed at an end of the casing, and a system board installed in the casing and electrically connected to at least one microcontroller and a power supply device; an end frame device having an end frame casing and a patch installed under the end frame; a stretching device having a stretching member with an end inserted into the casing port and the other end connected to the end frame casing; a position sensor installed on the system board and electrically connected to a microcontroller; and at least one warning device electrically connected the microcontroller, so that if a wearer's spine is in an improper curvature condition, a warning message will be issued to remind the wearer to adjust the posture and prevent spinal disease and injury.

### TECHNICAL FIELD

The technical field relates to a spine examination apparatus, and more particularly to the apparatus capable of detecting a person's improper spinal curvature condition to generate an appropriate warning to warn such person.

### BACKGROUND

Lumbar spine refers to the vertebra disposed at the middle of a back of a person, and the spine is a support column of a human body to support, protect and balance the human body and allow movements. Nowadays, most people ignore the fundamental importance of spinal healthcare, and thus often offer intolerable pain caused by spinal diseases or even lose the capacity to work. For instance, people nowadays habitually watch television while sitting in a couch with incorrect posture, use 3C products while bending down and lowering head, or read a book, watch TV or use a mobile phone in bed, and such long-term incorrect sitting, standing and sleeping postures may cause spinal diseases and symptoms such as back pain, slipped disc, sciatica, osteoporosis, compression fracture, etc, and these diseases causes all kinds of pain. In a mild case, a patient just begins with a feeling of discomfort or inconvenient to move, but in a serious case, the patient's daily life and work may be affected or such diseases may even lead to paralysis. For example, the symptoms of scoliosis and nerve compression may cause back pain, and the patient may feel like an electric current passing through both legs and suffer severe pain, and thus causing poor and slow walking postures or being ill at ease, etc.

Present physical treatments for the related spinal diseases include treatment apparatuses and braces, but these apparatuses are mainly intended for correcting or treating spinal problems instead of preventing spinal diseases or merging the concept of prevention into daily life. For example, an improper spinal posture reminder is capable of reminding a wearer about an improper spinal posture timely to improve and correct the wearer's bad posture immediately and such apparatus can be used to educate adolescents and children to develop correct postures and prevent bad posture at an early stage. In the meantime, the apparatus can also discover any spinal problem to prevent the problem from getting worse at the early stage, so as to prevent spinal diseases from affecting children's academic learning or losing self-confidence. Therefore, it is a main subject for related manufactures to overcome the aforementioned problems and provide an effective improper spinal posture reminder.

In view of the aforementioned problems of the conventional spinal treatment apparatus and brace, the discloser of this disclosure conducted extensive researches and experiments, and finally developed improper spinal curvature warning apparatus with good detection, timeliness and alert.

### SUMMARY

Therefore, it is a primary objective of this disclosure to provide an improper spinal curvature warning apparatus capable of reminding a wearer about an improper spinal curvature and timely issuing an alert to remind the wearer to adjust the posture and prevent developing any bad posture that will progressively produce a pathological change or injury.

Another objective of this disclosure is to provide an improper spinal curvature warning apparatus, such that a wearer may transmit, monitor, analyze, and track the information and status of the wearer's spine in daily life, so that the wearer may take proper actions for the correct spinal healthcare, correction and management.

A further objective of this disclosure is to provide an improper spinal curvature warning apparatus capable of facilitating a wearer to make adjustments to the warning application according to different environmental conditions and automatically apply an appropriate physical therapy to the spine, so as to achieve the dual effects of reminding an improper spinal curve and providing a timely physical therapy.

To achieve the aforementioned and other objectives, this disclosure provide an improper spinal curvature comprising: a main body, including a casing and a patch installed at the bottom of the casing, an end of the casing having a casing port formed thereon, and the casing having a system board installed therein and electrically coupled to at least one microcontroller and a power supply device; an end frame device, including an end frame casing and patch installed under the end frame casing; a stretching device, including a stretching member with an end inserted into the casing port and the other end coupled to the end frame casing; a position sensor, electrically coupled to the microcontroller; and at least one warning device, electrically coupled to the microcontroller.

In the aforementioned assembly, the casing has a distal extended tube portion extended from the casing and provided for forming the casing port.

In the aforementioned assembly, wherein the stretching device includes a position restoring member having an end fixed to the system board or the casing and the other end fixed to the stretching member.

In the aforementioned assembly, the end frame casing have an internal port and an external port formed at both ends of the end frame casing respectively for inserting the stretching member, and the internal port and the casing port are arranged opposite to each other, and the end frame casing has a positioning button disposed thereon and a positioning member extended downwardly from the positioning button.

In the aforementioned assembly, wherein the stretching member has a plurality of holes disposed opposite to a moving area of the end frame casing, and the plurality of holes is a plurality of separate positioning holes for latching the positioning member.

In the aforementioned assembly, wherein the position sensor is a varistor, and the varistor includes a variable resistor member and a slide member, and the variable resistor member is installed on the system board and both ends of the variable resistor member are a first fixed connecting end and a second fixed connecting end, and the first fixed connecting end and the second fixed connecting end are electrically coupled to the system board, and the slide member is slidably installed on the variable resistor member, and the slide member has a slidiably connecting end coupled to the stretching member.

In the aforementioned assembly, wherein the position sensor includes a first limit member and a second limit member disposed on an inner side of the positioning sensor and arranged opposite to the first fixed connecting end and the second fixed connecting end respectively.

In the aforementioned assembly, wherein the position sensor is an encoder including a conductive plate and a gear, and the conductive plate includes a conductive gear disposed thereon and engaged with the conductive gear, and the stretching member has a toothed section disposed on a side of the stretching member and opposite to the gear, and the toothed section is engaged with the gear.

In the aforementioned assembly, wherein the warning device includes a buzzer, a vibration motor or a pulse generator.

In the aforementioned assembly, wherein the microcontroller is electrically coupled to a control key module provided for selecting a function and performing a control operation.

In the aforementioned assembly, wherein the system board further includes a communication module electrically coupled to the microcontroller for connecting a portable electronic device for a wireless communication.

This disclosure will become clearer in light of the following detailed description of an illustrative embodiment of this invention described in connection with the drawings.

### BRIEF DESCRIPTION OF THE DRAWINGS

FIG. 1 is a schematic view of an application of a first preferred embodiment of this disclosure;

FIG. 2 is a schematic view of a structure of the first preferred embodiment of this disclosure;

FIG. 3 is a schematic view of an operation of the first preferred embodiment of this disclosure;

FIG. 4 is a bottom view of the first preferred embodiment of this disclosure;

FIG. 5 is a side view of the first preferred embodiment of this disclosure;

FIG. 6 is a schematic view of a structure of a second preferred embodiment of this disclosure; and

FIG. 7 is a schematic view of a circuit system and a transmission application of this disclosure.

### DESCRIPTION OF THE PREFERRED EMBODIMENTS

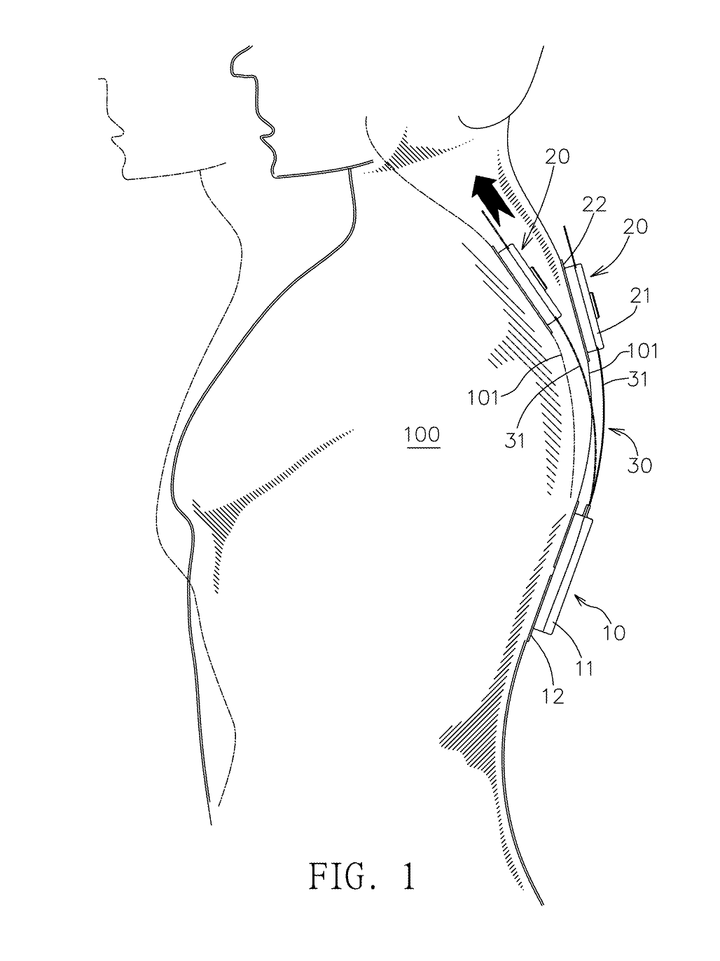

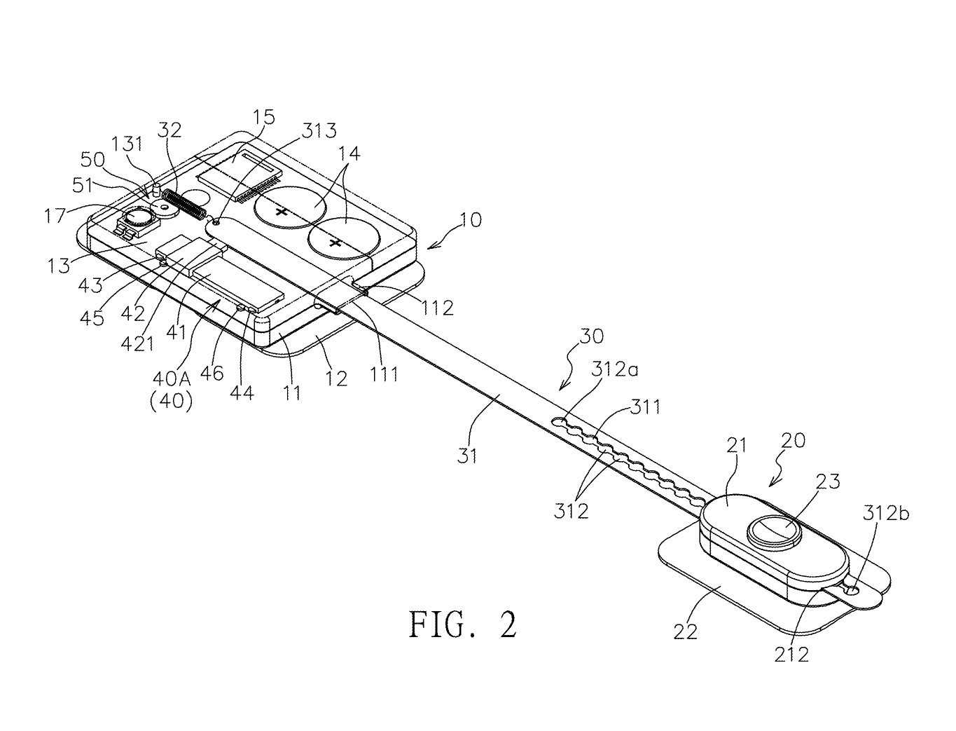

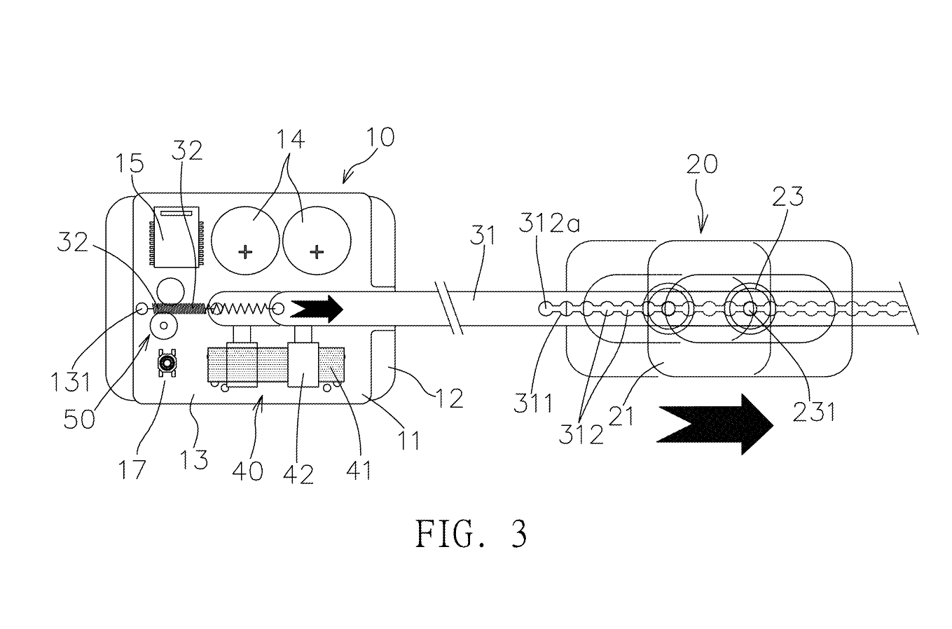

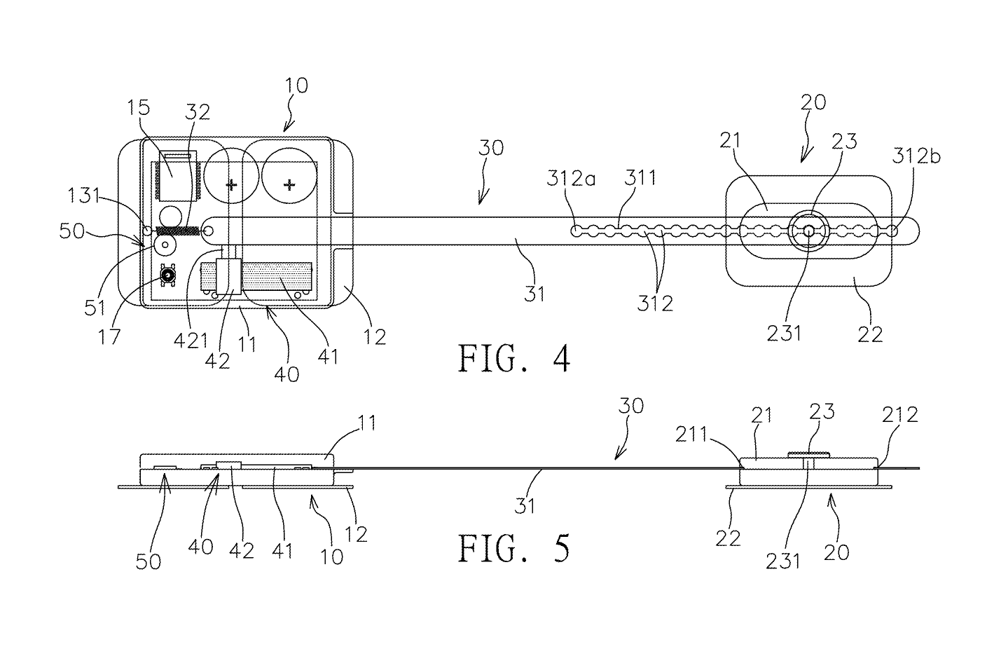

With reference to FIGS. 1 to 5 for an improper spinal curvature warning apparatus in accordance with the first preferred embodiment of this disclosure, these figures are simplified schematic figures provided for illustrating the basic structure of this disclosure, and the structures as shown in the figures are not drawn according to the shape, size and proportion for the actual apparatus. These figures are intended for the illustration of this disclosure, but not for limiting the scope of this disclosure. The improper spinal curvature warning apparatus of this disclosure comprises a main body 10, an end frame device 20, a stretching device 30, a position sensor 40 and a warning device 50. The main body 10 includes a casing 11 and a first patch 12 (or a patch) installed under the casing 11, and the casing 11 has a casing port 111 disposed at an end of the casing 1, and a distal extended tube portion 112 is extended from the casing 11 in this preferred embodiment, and the casing port 111 is disposed at an end of the distal extended tube portion 112 and communicated with the interior of the casing 11. The casing 11 has a system board 13 installed therein, wherein the system board 13 is a printed circuit board electrically coupled to at least one microcontroller 15 and related electronic components (not shown in the figure) and the microcontroller of this preferred embodiment is installed on the system board 13. The system board 13 includes a connecting column 131 and a power supply device 14 installed thereon, and the power supply device 14 is a battery (or a rechargeable battery) for supplying the required electric power for the operation of the apparatus of this disclosure. In addition, the microcontroller 15 is electrically coupled to a control key module 17, wherein the control key module 17 of this embodiment is installed on a surface of the casing 11 to facilitate users to select the using functions and perform the control operations. The end frame device 20 includes an end frame casing 21 and a second patch 22 (or a patch) installed under the end frame casing 21, and both ends of the frame casing 21 have an internal port 211 (as shown in FIG. 5) and an external port 212 respectively, and the internal port 211 and the external port 212 are communicated with the interior of the end frame casing 21, and the internal port 211 and the casing port 111 are arranged opposite to each other, and the end frame casing 21 includes a positioning button 23 disposed thereon, a positioning member 231 extended downwardly from the positioning button 23 (as shown in FIG. 3, and the positioning member and the positioning button of this preferred embodiment are perpendicular to each other).

The stretching device 30 includes a stretching member 31 and a position restoring member 32, wherein the stretching member 31 is a plate coupled between the main body 10 and the end frame device 20, and the stretching member 31 has an end inserted into the casing port 111 of the casing 11 and the other end inserted into the internal port 211 of the end frame casing 21, and a connecting hole 313 is formed at an end of the casing 11 for inserting the stretching member 31. The stretching member 31 is inserted into an end of the end frame casing 21 and passed out from the external port 212, but this disclosure is not limited by the aforementioned design. The stretching member 31 has a plurality of holes 311 disposed opposite to the moving area of the end frame casing 21, wherein the plurality of holes 311 is a plurality of separate positioning holes 312, and the positioning holes 312 are provided for latching the positioning member 231 to position the stretching member 31, so that the positioning hole 312 may be selected and positioned to adjust and set the range of the using area of the main body 10 and the attached end frame device 20, and the plurality of positioning holes 312 (or the plurality of holes 311) has a front positioning hole 312a and a rear positioning hole 312b acting as end limits for the adjustment of the stretching member 31. The position restoring member 32 may be a spring, and both ends of the position restoring member 32 are coupled to the connecting column 131 (or the casing 11) and the connecting hole 313 (or the stretching member 31) respectively, so that the stretching member 31 is positioned at an end of the casing 11, and a displaced stretching member 31 may resume its original position by the resilience of the position restoring member 32.

The position sensor 40 is installed on the system board 13 in the casing 11. In this preferred embodiment, the position sensor 40 is a varistor 40A (or a variable resistor, VR), wherein the position sensor 40 (or the varistor 40A) includes a variable resistor member 41 and a slide member 42, and the variable resistor member 41 is installed on the system board 13 and electrically coupled to the system board 13 and the microcontroller 15, and both ends of the variable resistor member 41 are a first fixed connecting end 43 and a second fixed connecting end 44, and both of the first fixed connecting end 43 and the second fixed connecting end 44 are electrically coupled to the system board 13. The slide member 42 is slidably installed on the variable resistor member 41, and the slide member 42 has a slidably connecting end 421 coupled to the stretching member 31, so that the slidably connecting end 421 (or the slide member 42) may be displaced together with the stretching member 31, and the sliding of the slidably connecting end 421 (or the slide member 42) changes the resistance between the slidably connecting end 421 and the first fixed connecting end 43 and the second fixed connecting end 44 or forms different voltage division ratios or detects a change of electric potentials at the sliding point in order to calculate the amount of displacement (or determine a curved status) of the stretching member 31. In addition, the position sensor 40 includes a first limit member 45 and a second limit member 46 disposed on an inner side of the position sensor 40 and opposite to the first fixed connecting end 43 and the second fixed connecting end 44 respectively for limiting the moving area of the slide member 42 and protecting the first fixed connecting end 43 and the second fixed connecting end 44. The warning device 50 is installed on the system board 13 and electrically coupled to the system board 13 and the microcontroller 15, and the warning device 50 includes a sound generator 51 (such as a speaker, a buzzer, etc), for generating a warning sound.

When the improper spinal curvature warning apparatus of this disclosure is used, the first patch 12 and the second patch 22 are attached onto a spine and back area 101 of a human body 100. Now, the stretching member 31 is positioned by the positioning member 231 and fixed to an appropriate length (or status), so that if the spine and back area 101 is bent, the stretching member 31 will be bent accordingly to pull out the casing 11, and the position restoring member 32 will be pulled and stretched, and the slidably connecting end 421 (or the slide member 42) will be displaced together with the stretching member 31. The slidably connecting end 421 (or the slide member 42) on the variable resistor member 41 shifts its position, so that the position sensor 40 generates a resistance, current or voltage change signal which is transmitted to the microcontroller 15 to calculate the amount of displacement of the stretching member 31. If the amount of displacement of the stretching member 31 is greater than a set value, the microcontroller 15 will turn on the warning device 50 and drive the sound generator 51 to issue a warning sound to remind a wearer about the posture with an improper spinal curvature and to timely adjust the incorrect posture. When the wearer has adjusted the spinal curvature posture, the stretching member 31 is partially pulled into the casing 11 or resumes its original status. In other words, when the stretching member 31 is displaced, the position sensor 40 senses the amount of displacement of the stretching member 31 and transmits a message of the amount of displacement of the stretching member 31 to the microcontroller 15. If the amount of displacement of the stretching member is equal to or greater than a numeric value set by the microcontroller 15, the microcontroller 15 will transmit a warning message to the warning device 50 and drive the warning device 50 to issue a warning (such as a sound, a vibration or an electrical stimulation physical therapy, etc).

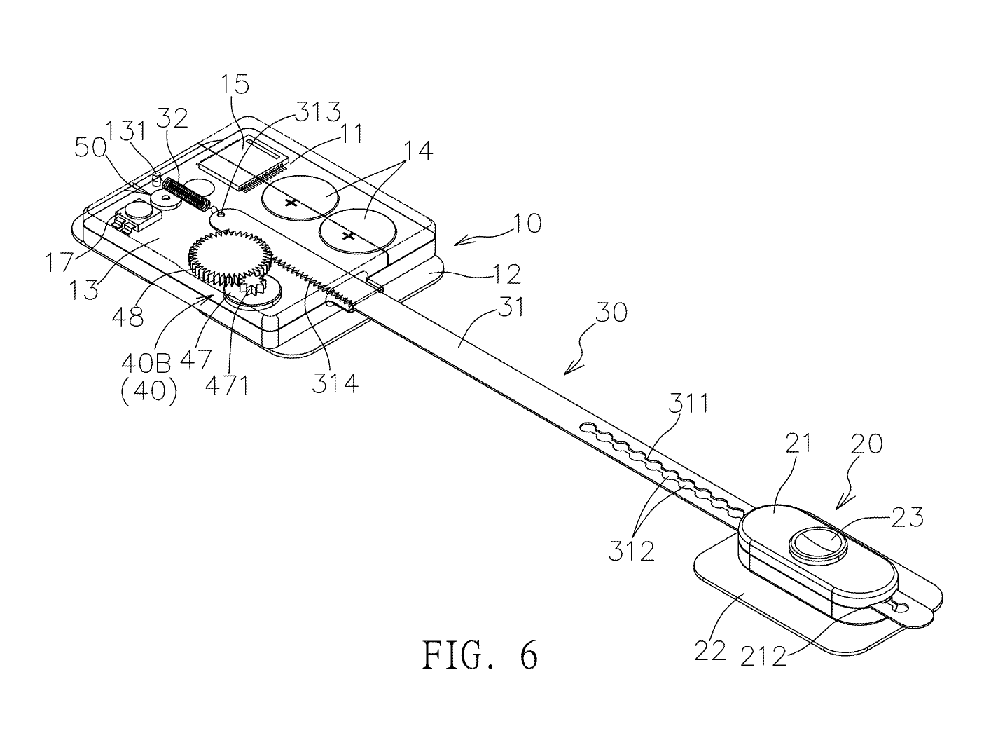

With reference to FIG. 6 for an improper spinal curvature warning apparatus in accordance with the second preferred embodiment of this disclosure, this embodiment is based on the basic structure of the first preferred embodiment to make modifications, and the difference between the first and second preferred embodiments resides on that the position sensor 40 of the second preferred embodiment is an encoder 40B, and the encoder 40B includes a conductive plate 47 and a gear 48, and the conductive plate 47 has a conductive gear 471. The gear 48 is engaged with the conductive gear 471, and the stretching member 31 has a toothed section 314 disposed on a side of the stretching member 31 and opposite to the gear 48, and the toothed section 314 and the gear 48 are engaged with each other. In other words, the stretching member 31 is linked to the gear 48 through the toothed section 314. When the spine and back area 101 is bent, the stretching member 31 is displaced together with the curvature and linked with the gear 48 and the conductive gear 471, so that the conductive plate 47 produces a rotary displacement, and the conductive plate 47 is provided for detecting a change of position by a mechanical contact (or a method of optics or magnetism), and the detected change of position is uses as a feedback signal for controlling the position. After the position is converted into an electronic signal, the electronic signal is transmitted to the microcontroller 15. If the amount of displacement of the stretching member 31 is greater than a predetermined value, then the microcontroller 15 will turn on the warning device 50 to issue a warning sound to remind a wearer about the posture with an improper spinal curvature and to adjust the incorrect posture.

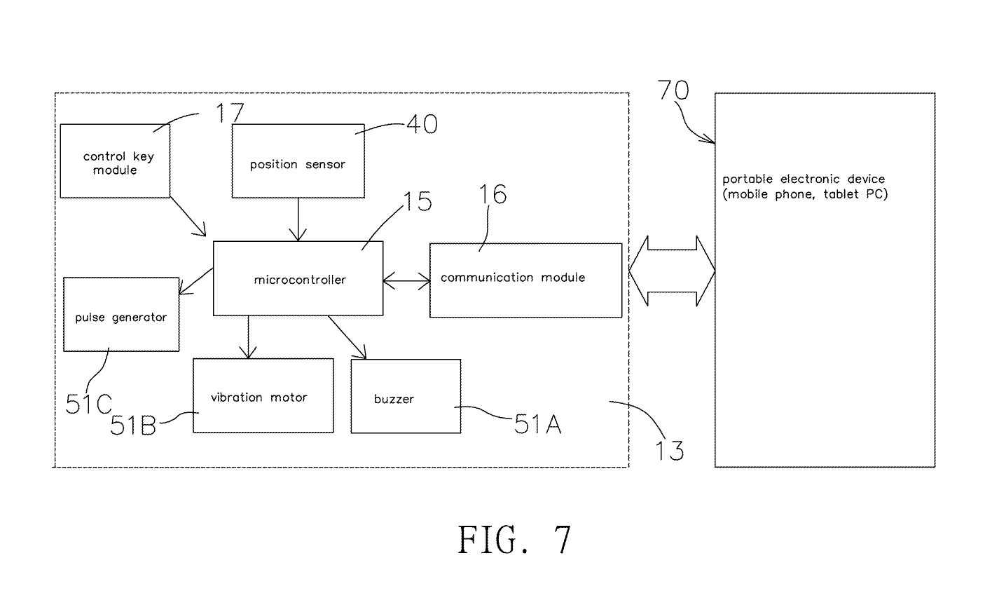

With reference to FIG. 7 for a circuit system and a transmission application of an improper spinal curvature warning apparatus in accordance with this disclosure, the microcontroller 15 installed on the system board 13 is electrically coupled to the position sensor 40 for receiving a message of a change of position of the stretching member 31, the warning device 50 electrically coupled to the microcontroller 15 includes a buzzer 51A, a vibration motor 51B or a pulse generator 51C (Pulse out), wherein, the buzzer 51A is provided for generating a sound warning, and the vibration motor 51B is provided for generating a vibration warning, and these warning devices may be selected and used according to different environments, and the pulse generator 51C not just has the warning effect only, but also provides physical therapy. In addition, the microcontroller 15 may be electrically coupled to a control key module 17, and the control key module 17 is provided for users to select a function and perform a control operation.

The system board 13 may further comprise a communication module 16 (such as a Bluetooth module) electrically coupled to the microcontroller 15, and a portable electronic device 70 (such as a mobile phone, a tablet PC, a notebook, etc) may be connected to the communication module 16 for a wireless communication, and the portable electronic device 70 may receive and display related messages and control operations through a program design or APP download, so that the wearer can transmit information of the spine status in daily life, and the spine condition and information can be fully monitored, analyzed, and kept track to facilitate related spinal healthcare and correction management and application. The apparatus of this disclosure is very convenient and useful.

In summation of the description above, the improper spinal curvature warning apparatus of this disclosure is capable of issuing a warning message to remind the wear about an improper curvature condition of the wearer's spine to adjust the incorrect posture, and prevent the wearer from developing the habit of having a bad posture or progressively generating a pathological change or being injured. In the meantime, the apparatus of this disclosure allows users to adjust the application for different environmental conditions and provides an appropriate physical therapy to the user's spine automatically. Therefore, the apparatus of this disclosure has due functions of reminding the users about an improper spinal curvature and timely providing a physical therapy.

What is claimed is:

1. An improper spinal curvature warning apparatus, attached onto a spine and back area of a human body, comprising:
a main body, attached to an end of the spine and back area of the human body, and including a casing and a patch installed at the bottom of the casing, a system board installed in the casing and electrically coupled to at least one microcontroller, and a power supply device;
an end frame device, attached to the other end of the main body opposite to the spine and back area of the human body, and including an end frame casing and a patch installed at the bottom of the end frame casing;
a stretching device, including a stretching member with an end inserted into the casing and the other end coupled to the end frame casing, and the stretching device producing a displacement and a deformation according to a spinal curvature of the human body spinal curvature;
a position sensor, electrically coupled to a microcontroller, for connecting the stretching member or sensing the displacement and deformation of the stretching member to generate an electric change signal;
at least one warning device, electrically coupled to the microcontroller, for receiving an electric signal control of the microcontroller;
such that when the stretching member is displaced, the position sensor senses the amount of displacement of the stretching member, and transmits a message of the amount of displacement of the stretching member to the microcontroller, and if the amount of displacement of the stretching member is equal to or greater than a numeric value set by the microcontroller, the microcontroller will transmit a warning message to the warning device, and the warning device will issue a warning.

2. The improper spinal curvature warning apparatus according to claim 1, wherein the casing has a distal extended tube portion extended from an end of the casing, and an end of the distal extended tube portion is provided for forming a casing port, and the casing port is provided for inserting an end of the stretching member, and the casing port is communicated with the interior of the casing.

3. The improper spinal curvature warning apparatus according to claim 2, wherein the end frame casing has internal port and an external port formed at both ends of the end frame casing respectively for inserting the stretching member, and the internal port and the casing port are disposed opposite to each other, and the end frame casing includes a positioning button installed thereon, and a positioning member extended downwardly from the positioning button.

4. The improper spinal curvature warning apparatus according to claim 3, wherein the stretching member has a plurality of holes disposed opposite to a moving area of the end frame casing, and the plurality of holes is a plurality of separate positioning holes for latching the positioning member.

5. The improper spinal curvature warning apparatus according to claim 1, wherein the position sensor is a varistor, and the varistor includes a variable resistor member and a slide member, and the variable resistor member is installed on the system board and both ends of the variable resistor member are a first fixed connecting end and a second fixed connecting end, and the first fixed connecting end and the second fixed connecting end are electrically coupled to the system board, and the slide member is slidably installed on the variable resistor member, and the slide member has a slidiably connecting end coupled to the stretching member.

6. The improper spinal curvature warning apparatus according to claim 5, wherein the position sensor includes a first limit member and a second limit member disposed on an inner side of the positioning sensor and arranged opposite to the first fixed connecting end and the second fixed connecting end respectively.

7. The improper spinal curvature warning apparatus according to claim 1, wherein the position sensor is an encoder including a conductive plate and a gear, and the conductive plate includes a conductive gear disposed thereon and engaged with the conductive gear, and the stretching member has a toothed section disposed on a side of the stretching member and opposite to the gear, and the toothed section is engaged with the gear.

8. The improper spinal curvature warning apparatus according to claim 1, wherein the warning device includes a buzzer, a vibration motor or a pulse generator.

9. The improper spinal curvature warning apparatus according to claim 1, wherein the microcontroller is electrically coupled to a control key module provided for selecting a function and performing a control operation.

10. The improper spinal curvature warning apparatus according to claim 1, wherein the system board further includes a communication module electrically coupled to the microcontroller for connecting a portable electronic device for a wireless communication.

11. The improper spinal curvature warning apparatus according to claim 1, wherein the stretching device further includes a position restoring member having an end fixed to the system board or the casing and the other end coupled to the stretching member, so that the stretching member is positionable at an end of the casing and the replaced stretching member is restorable to its original position by the resilience of the position restoring member.

12. The improper spinal curvature warning apparatus according to claim 11, wherein the position restoring member is a spring.
